(19) United States
(12) Patent Application Publication
(10) Pub. No.: US 2015/0094430 A1
(43) Pub. Date: Apr. 2, 2015

(54) DINITRO COMPOUND, DIAMINE COMPOUND, AND AROMATIC POLYIMIDE

(71) Applicant: TAIWAN TEXTILE RESEARCH INSTITUTE, NEW TAIPEI CITY (TW)

(72) Inventor: Wen-Hsiang Chen, TU-CHEN CITY (TW)

(73) Assignee: TAIWAN TEXTILE RESEARCH INSTITUTE, NEW TAIPEI CITY (TW)

(21) Appl. No.: 14/246,269

(22) Filed: Apr. 7, 2014

(30) Foreign Application Priority Data
Sep. 30, 2013 (TW) 102135362

(51) Int. Cl. C08G 73/10; C07C 217/90; C07C 205/38

(52) U.S. Cl. CPC C08G 73/1067; C07C 205/38; C07C 217/90
USPC 525/436; 568/586; 564/430

(57) ABSTRACT

A dinitro compound I, a diamine compound II and polyimides are provided. The diamine compound II is a reduction product of the dinitro compound I. The polyimides using the diamine compound II as one of the monomers can increase the solubility of the polyimides in various organic solvents and make the color of the polyimides to be transparent and colorless.

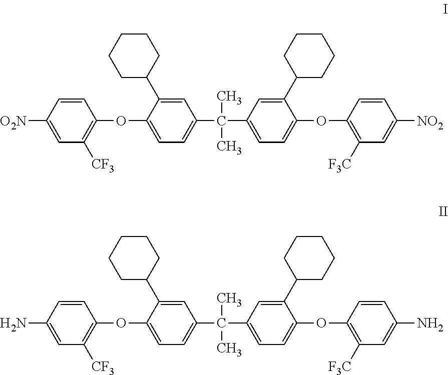 [SMILES: CC(C)(c1ccc(Oc2ccc([N+](=O)[O-])cc2C(F)(F)F)c(C2CCCCC2)c1)c1ccc(Oc2ccc([N+](=O)[O-])cc2C(F)(F)F)c(C2CCCCC2)c1 ; CC(C)(c1ccc(Oc2ccc(N)cc2C(F)(F)F)c(C2CCCCC2)c1)c1ccc(Oc2ccc(N)cc2C(F)(F)F)c(C2CCCCC2)c1]

### CROSS-REFERENCE TO RELATED APPLICATION

This application claims the priority benefit of Taiwanese application serial no. 102135362, filed Sep. 30, 2013, the full disclosure of which is incorporated herein by reference.

### BACKGROUND

1. Technical Field

The disclosure relates to a polyimide and a preparation method thereof. More particularly, the disclosure relates to a polyimide with pale color and a preparation method thereof.

2. Description of Related Art

Polyimide is a common engineering plastic. Since polyimide has properties of wide applicable temperature range, excellent chemical resistance, and high mechanical strength, the polyimide has a wide application range. Although aromatic polyimide has good thermal stability, the solubility of the aromatic polyimide, which is polymerized by aromatic diamine and aromatic tetracarboxylic dianhydride, in most common organic solvents is very poor, and some aromatic polyimide even only may be dissolved in concentrated sulfuric acid. Therefore, the aromatic polyimide cannot be easily processed. Moreover, since intermolecular or intramolecular charge transfer is easily occurred in aromatic polyimide to form charge transfer complex (CTC), most aromatic polyimides have a deep color. Therefore, the application on optoelectronic products, such as flexible liquid crystal displays, color e-papers, organic light emitting diodes, organic photovoltaics or aerospace, of the aromatic polyimide is limited.

### SUMMARY

Accordingly in one aspect, the present disclosure provides a dinitro compound I having a chemical structure shown below.

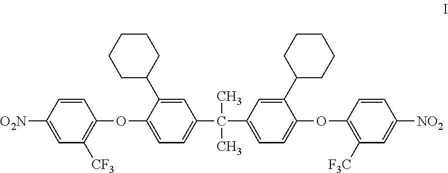 [SMILES: CC(C)(c1ccc(Oc2ccc([N+](=O)[O-])cc2C(F)(F)F)c(C2CCCCC2)c1)c1ccc(Oc2ccc([N+](=O)[O-])cc2C(F)(F)F)c(C2CCCCC2)c1]

In another aspect, the present disclosure provides a diamine compound II having a chemical structure shown below.

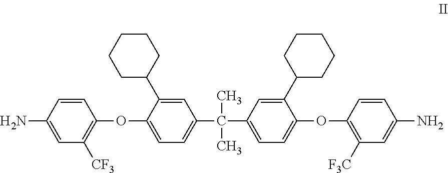 [SMILES: CC(C)(c1ccc(Oc2ccc(N)cc2C(F)(F)F)c(C2CCCCC2)c1)c1ccc(Oc2ccc(N)cc2C(F)(F)F)c(C2CCCCC2)c1]

In yet another aspect, the present disclosure provides a polyimide. Monomers of the polyimide comprises a first aromatic diamine monomer having a chemical structure of the diamine compound II shown below,

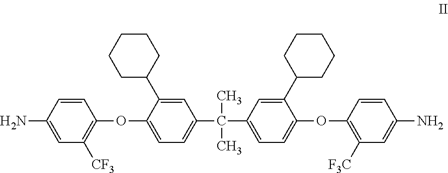 [SMILES: CC(C)(c1ccc(Oc2ccc(N)cc2C(F)(F)F)c(C2CCCCC2)c1)c1ccc(Oc2ccc(N)cc2C(F)(F)F)c(C2CCCCC2)c1]

and a tetracarboxylic dianhydride monomer having a chemical structure of

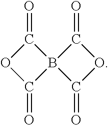

The B in the chemical structure of the tetracarboxylic dianhydride monomer may be

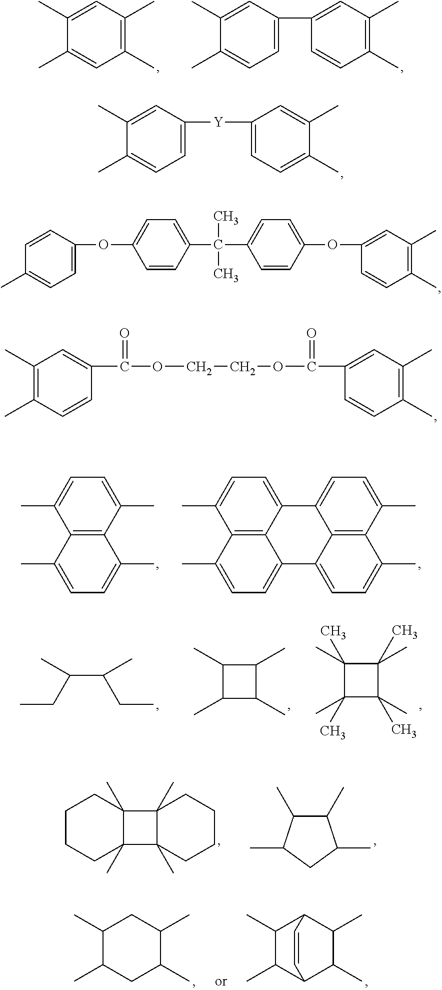

for example.

According to an embodiment, the above

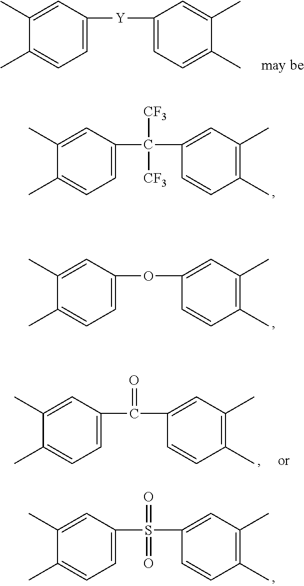

for example.

In another embodiment, the monomers of the polyimide further comprises a second aromatic diamine monomer having a chemical structure of H₂N-A-NH₂, and A may be

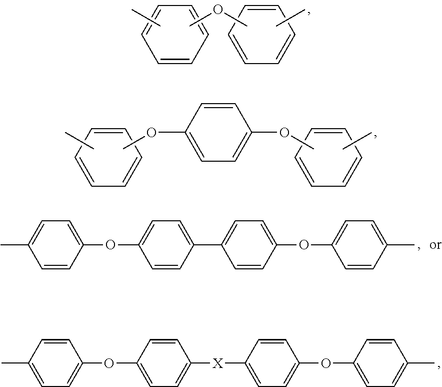

for example.

In yet another embodiment, the above

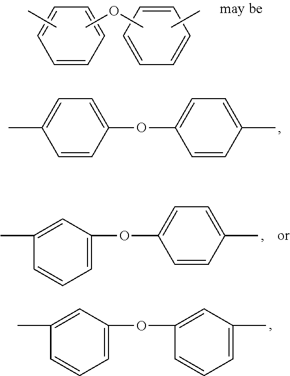

for example.

In yet another embodiment, the above

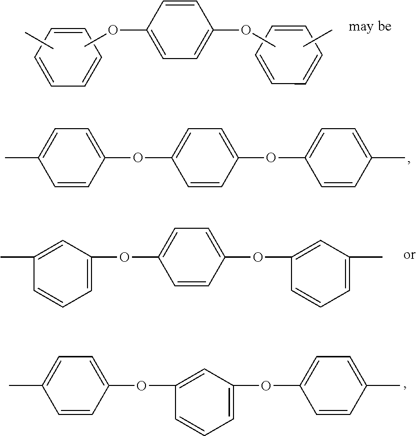

for example.

In yet another embodiment, the above

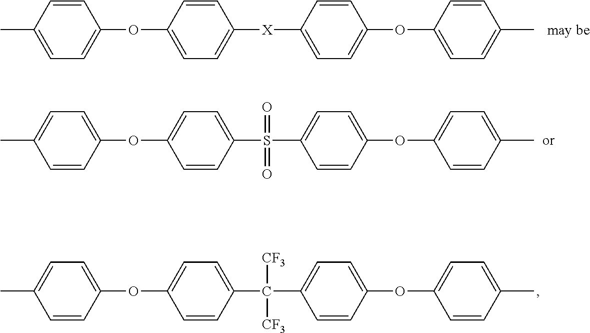

for example.

In yet another embodiment, a molar ratio of the second aromatic diamine monomer to the first aromatic diamine monomer is 0-99.

In yet another embodiment, the monomers of the polyimide further comprises 4,4′-diamino diphenyl ether.

It is to be understood that both the foregoing general description and the following detailed description are by examples, and are intended to provide further explanation of the disclosure as claimed.

The foregoing presents a simplified summary of the disclosure in order to provide a basic understanding to the reader. This summary is not an extensive overview of the disclosure and it does not identify key/critical elements of the present disclosure or delineate the scope of the present disclosure. Its sole purpose is to present some concepts disclosed herein in a simplified form as a prelude to the more detailed description that is presented later. Many of the attendant features will be more readily appreciated as the same becomes better understood by reference to the following detailed description considered in connection with the accompanying drawings.

### BRIEF DESCRIPTION OF THE DRAWINGS

FIGS. 1A-1C are ¹H NMR, ¹³C NMR, and IR spectra of the dinitro compound I, respectively.

FIGS. 2A-2C are ¹H NMR, ¹³C NMR, and IR spectra of the diamine compound II, respectively.

FIGS. 3A-3C are ¹H NMR, ¹³C NMR, and IR spectra of the polyimide PMDA-100, respectively.

FIG. 4 is IR spectrum of 6FDA-100.

### DETAILED DESCRIPTION

Accordingly, a polyimide with a pale color to colorless are provided. In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### Polyimide Using Diamine Compound II as a Diamine Monomer

A polyimide with pale color to colorless is provided. The monomers of the polyimide include an aromatic diamine monomer and a tetracarboxylic dianhydride monomer. The aromatic diamine monomer includes a first aromatic diamine monomer having a chemical structure of a diamine compound II shown below.

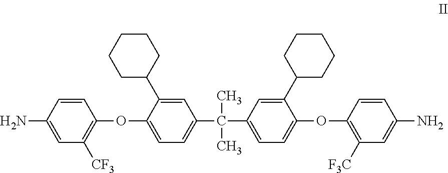 [SMILES: CC(C)(c1ccc(Oc2ccc(N)cc2C(F)(F)F)c(C2CCCCC2)c1)c1ccc(Oc2ccc(N)cc2C(F)(F)F)c(C2CCCCC2)c1]

In the diamine compound II above, trifluoromethyl and cyclohexyl groups are introduced onto the outer and inner benzene rings, respectively. Therefore, the steric regularity of the obtained polyimide may be decreased. This, in turn, is such that the molecular chains of the polyimide cannot be close to each other to be easily stacked together and thereby increase the solubility of the polyimide in polar organic solvents. The increased solubility makes the polyimide facilitate the coating process. Moreover, since the molecular chains of the polyimide cannot be easily close to each other to be stacked together, the phenomenon of intermolecular charge transfer may be decreased, such that the color of the polyimide may be decreased to pale color, or even colorless.

The tetracarboxylic dianhydride monomer above has a chemical structure of

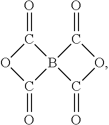

### and B is

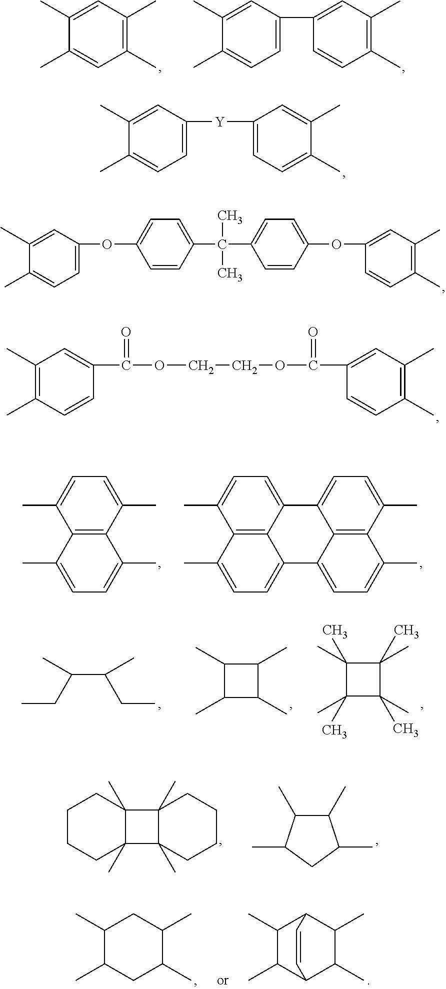

In some embodiments, the above

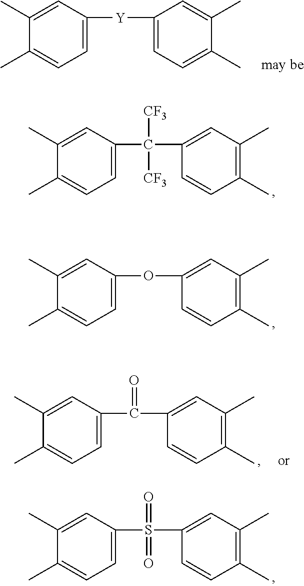

for example.

In some embodiments, the diamine monomer may further include a second aromatic diamine monomer, which has a chemical structure of H₂N-A-NH₂, and A may be

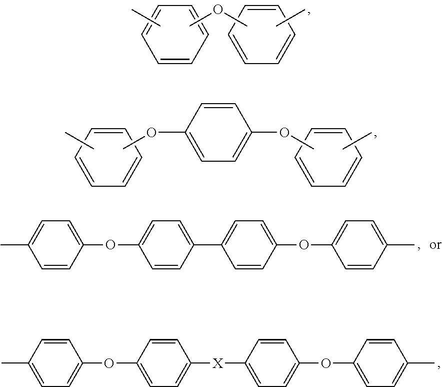

for example.

In some embodiments, the above

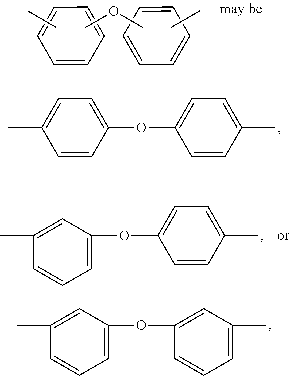

for example

In some other embodiments, the above

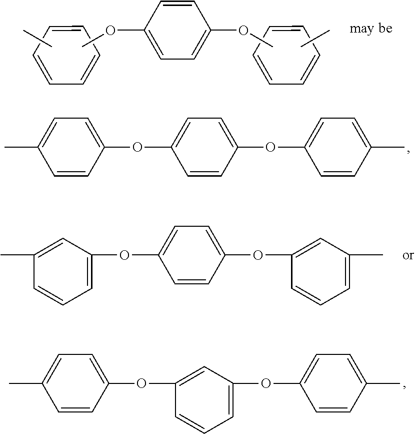

for example

In some other embodiments, the above

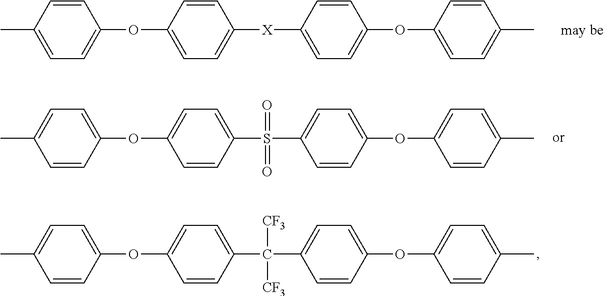

for example.

### Synthesis of Polyimide Using Diamine Compound II as One Diamine Monomer

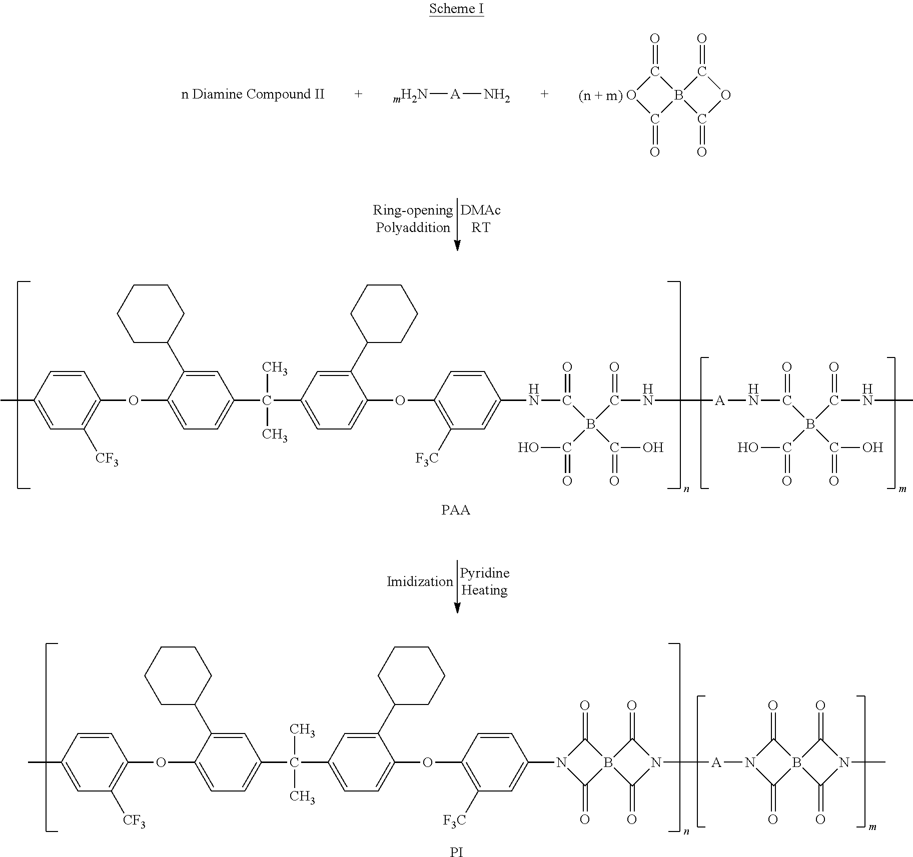

The synthesis method of the polyimide is shown in Scheme I. In the beginning, the first aromatic diamine monomer (i.e. the diamine compound II), optionally the second aromatic diamine monomer (H₂N-A-NH₂), and the tetracarboxylic dianhydride monomer are dissolved in dimethylacetamide (DMAc) to perform ring opening reaction and polyaddition reaction successively and in turn form an intermediate, i.e. polyamic acid (PAA). The total molar number of the first and the second aromatic diamine monomers is equal to the molar number of the tetracarboxylic dianhydride monomer. Next, a cyclization reaction of the polyamic acid is continuously performed to form polyimide (PI).

The molar ratio of the second aromatic diamine monomer to the first aromatic diamine monomer is 0 to about 99. The number of the polyimde unit composed of one diamine monomer and one tetracarboxylic dianhydride monomer is about 2-500.

### Embodiment 1

### Synthesis of Diamine Compound II

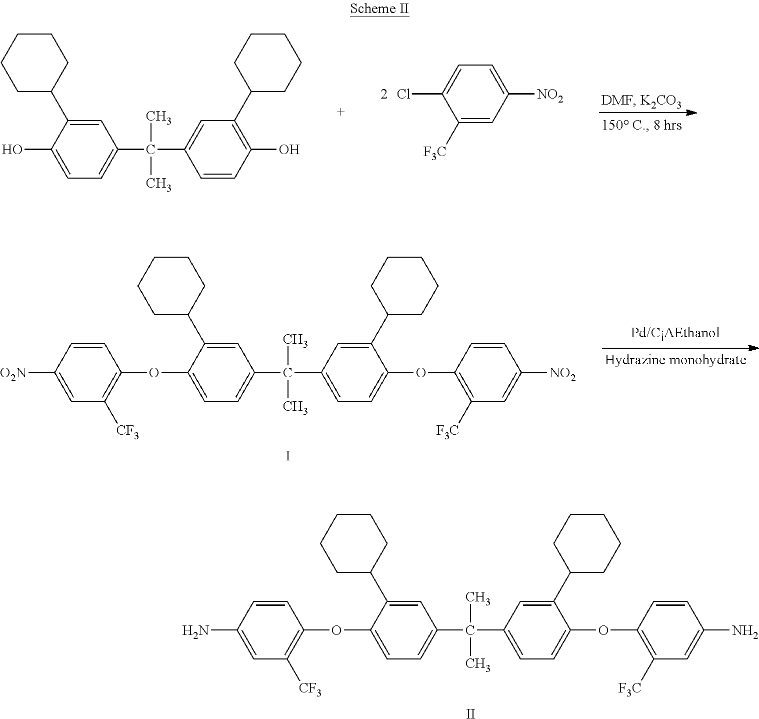

In this embodiment, the diamine compound II is synthesized first. In Scheme II, 2,2-bis(4-hydroxy-3-cyclohexylphenyl) propane and 2-chloro-5-nitrobenzotrifluoride were reacted at 150° C. for 8 hours to obtain the dinitro compound I above. Next, a reduction reaction was performed to reduce the dinitro compound I to obtain the diamine compound II.

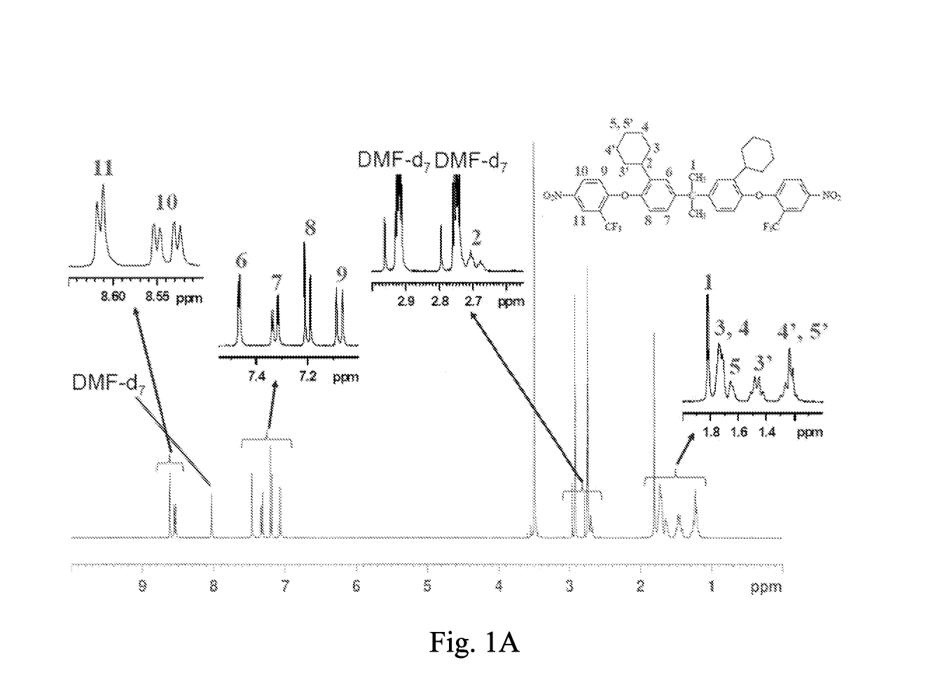

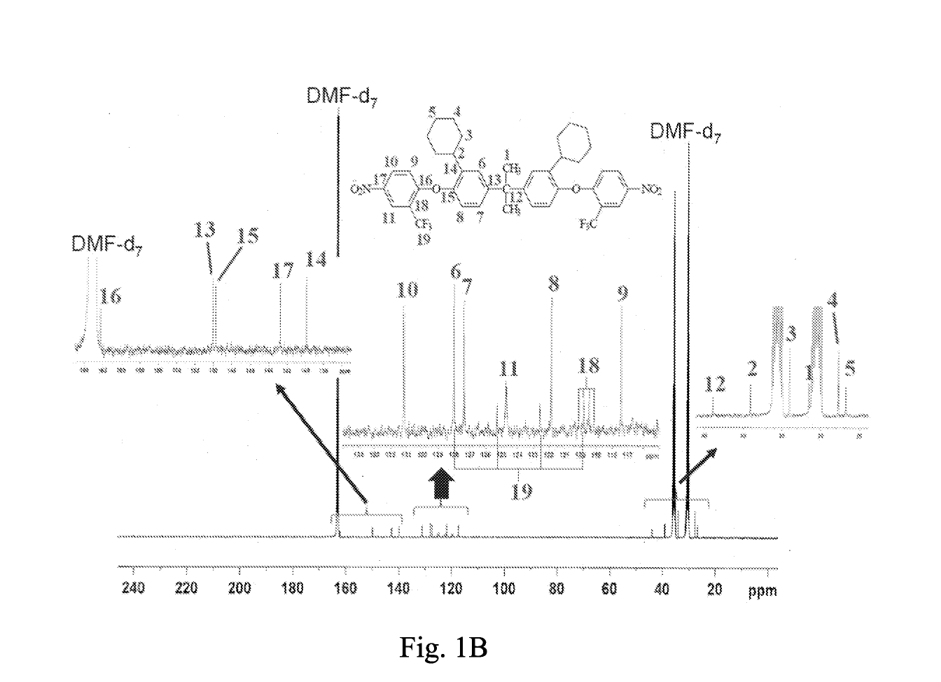

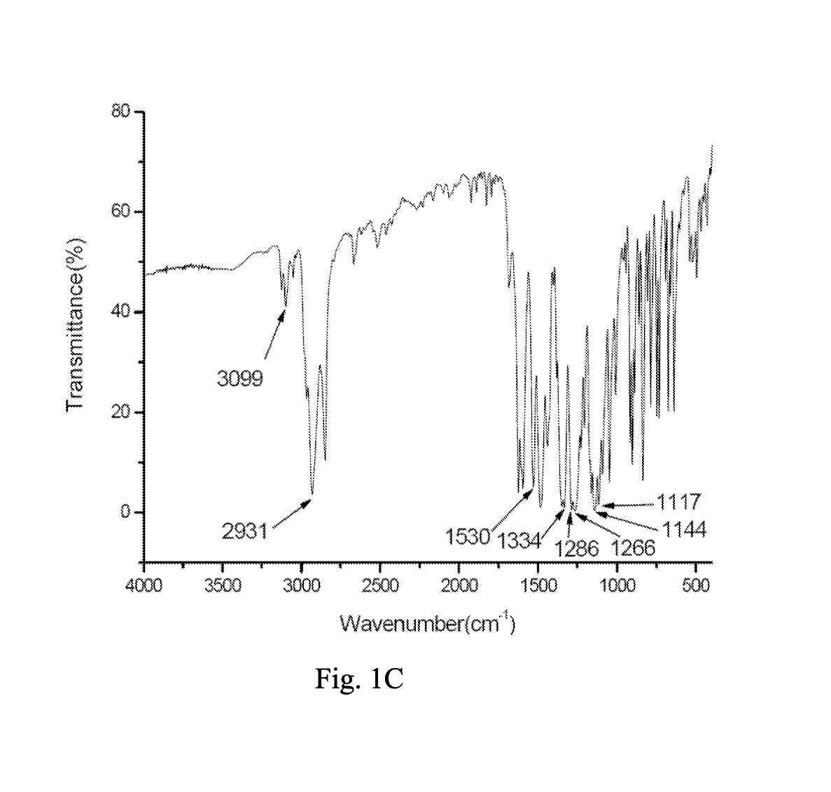

The detailed synthesis steps of the dinitro compound I are described below. 20 mmole of 2,2-bis(4-hydroxy-3-cyclohexylphenyl) propane, mmole of 2-chloro-5-nitrobenzotrifluoride, and 100 mL of N,N-dimethylformamide (DMF) are added into a reaction flask. The mixture was heated under a reflux condition and then reacted for 8 hours. After completion of the reaction, the reaction mixture was cooled down to room temperature. Next, the reaction mixture was poured into 500 mL of methanol, and then filtered to obtain yellow powder. The yellow powder was dried in an oven, and then purified by recrystallization in N,N-dimethylformamide to obtain the novel dinitro compound I with cyclohexyl and trifluoromethyl groups. The yield of the dinitro compound I was 85%. The melting point of the dinitro compound I was 243° C. FIGS. 1A-1C are ¹H NMR, ¹³C NMR, and IR spectra of the dinitro compound I, respectively. The other spectra data are listed below.

¹H-NMR (DMF-d₇): δ (ppm)=8.61 (2H, H₁₁), 8.55-8.53 (2H, H₁₀), 7.47 (2H, H₆), 7.34-7.32 (2H, H₇), 7.19 (2H, H₈), 7.07 (2H, H₉), 2.71 (2H, H₂), 1.82 (6H, H₁), 1.74 (8H, H₃ and H₄), 1.66 (2H, H₅), 1.47 (4H, H_{3′}), 1.23 (6H, H_{4′} and H_{5′}); ¹³C-NMR (DMF-d₇): δ (ppm)=162.4 (C₁₆), 150.1 (C₁₃), 149.8 (C₁₅), 142.8 (C₁₇), 139.9 (C₁₄), 131.2 (C₁₀), 128.0 (C₆), 127.4 (C₇), 124.7 (C₁₁), 127.9, 125.3, 122.6, 119.8 (C₁₉), 121.8 (C₈), 120.1, 119.8, 119.5, 119.1 (C₁₈), 117.4 (C₉), 44.0 (C₁₂), 39.1 (C₂), 34.1 (C₃), 31.5 (C₁), 27.7 (C₄), 26.8 (C₅). IR spectrum (cm⁻¹): 3099 (stretching vibration of aromatic C—H), 2931, and 2848 (stretching vibrations of aliphatic C—H), 1530 and 1334 (stretching vibrations of —NO₂), and 1286, 1266, 1144 and 1177 (stretching vibrations of C—F and C—O).

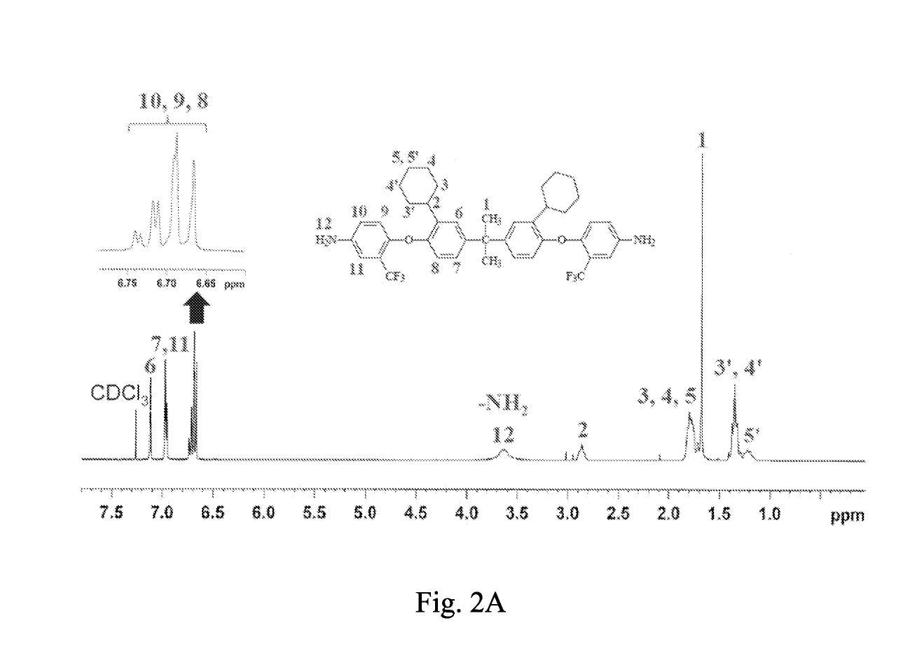

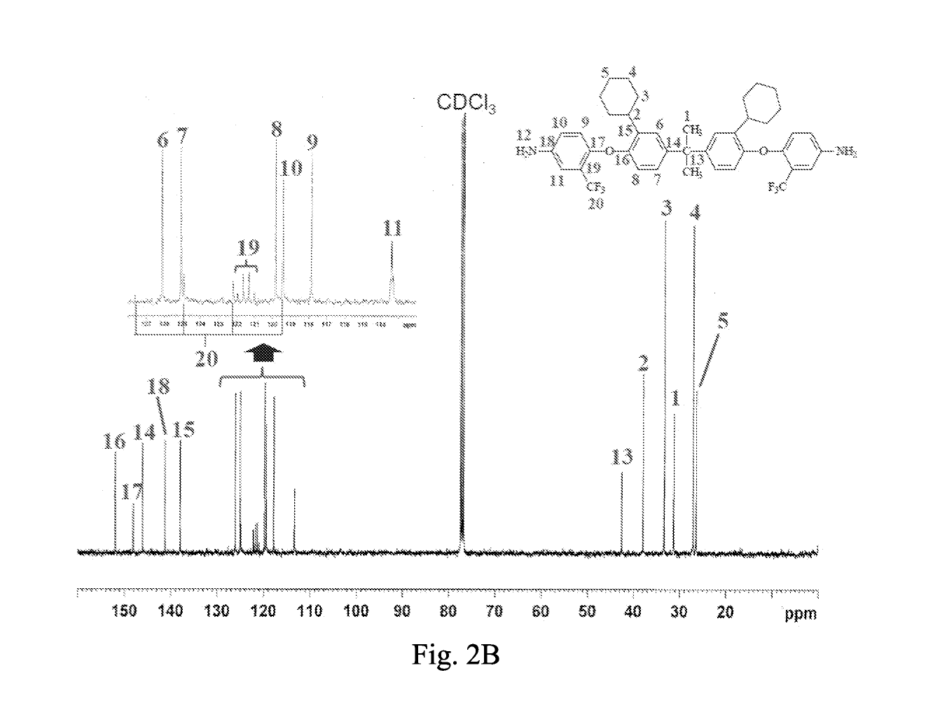

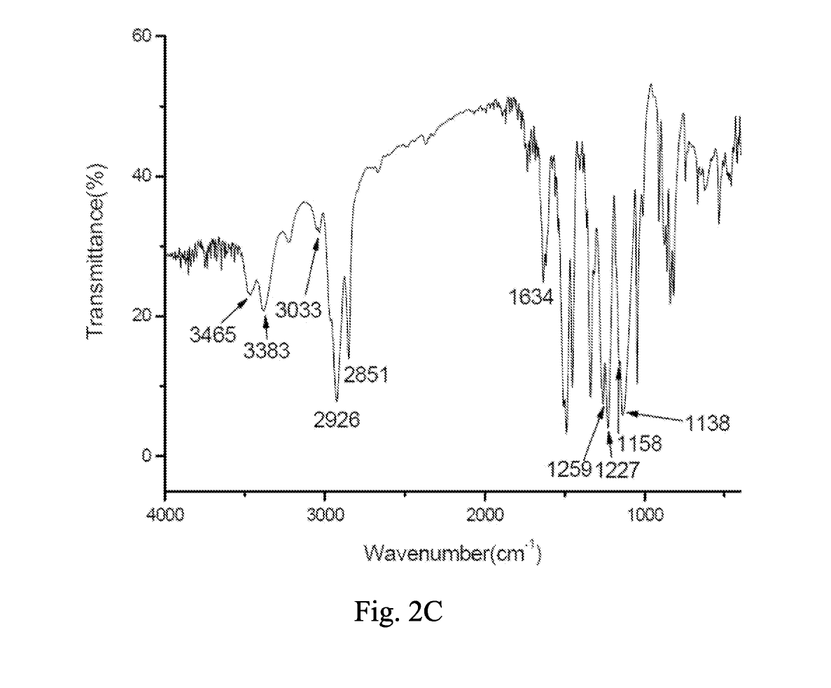

The detailed synthesis steps of the diamine compound II are described below. 10 g of dinitro compound I, 120 mL of ethanol, and 1 g of 10 wt % of palladium supported by carbon (Pd/C) catalyst were added into a two-neck flask to form a reaction mixture. After the reaction mixture was heated under a reflux condition, 10 mL of hydrazine monohydrate (H₂NNH₂.H₂O) was slowly dropped into the flask. The reduction reaction was conducted for 24 hours. Then, filtering was immediately performed to obtain white crystals of the diamine compound II. The yield of the diamine compound II was 90%. The melting point of the diamine compound II was 61° C. FIGS. 2A-2C are ¹H NMR, ¹³C NMR, and IR spectra of the diamine compound II, respectively. The other spectra data are listed below.

¹H-NMR (CDCl₃): δ (ppm)=7.12 (2H, H₆), 6.98˜6.95 (4H, C₇ and C₁₁), 6.74˜6.72 (2H, C₁₀), 6.69˜6.67 (4H, C₉ and C₈), 3.63 (4H, H₁₂), 2.87 (2H, H₂), 1.82˜1.76 (8H, H₃ and H₄), 1.71 (2H, H₅), 1.67 (6H, H₁), 1.41˜1.29 (8H, H_{3′} and H_{4′}), 1.22 (2H, H_{5′}); ¹³C-NMR (CDCl₃): δ (ppm)=151.9 (C₁₆), 148.1 (C₁₇), 146.1 (C₁₄), 141.2 (C₁₈), 137.9 (C₁₅), 126.1 (C₆), 125.1 (C₇), 127.6, 124.9, 122.2, 119.5 (C₂₀), 121.9, 121.6, 121.3, 121.0 (C₁₉), 119.8 (C₈), 119.4 (C₁₀), 117.8 (C₉), 113.4 (C₁₁), 42.6 (C₁₃), 37.9 (C₂), 33.3 (C₃), 31.2 (C₁), 27.0 (C₄), 26.4 (C₅). IR spectrum (cm⁻¹): 3465 (asymmetric stretching vibration of N—H), 3383 (symmetric stretching vibrations of N—H), 3033 (stretching vibration of aromatic C—H), 2926 and 2851 (stretching vibrations of aliphatic C—H), 1634 (bending vibration of —NH₂) and 1259, 1227, 1158 and 1138 (stretching vibrations of C—F and C—O).

### Embodiment 2

### Synthesis of Polyimide III

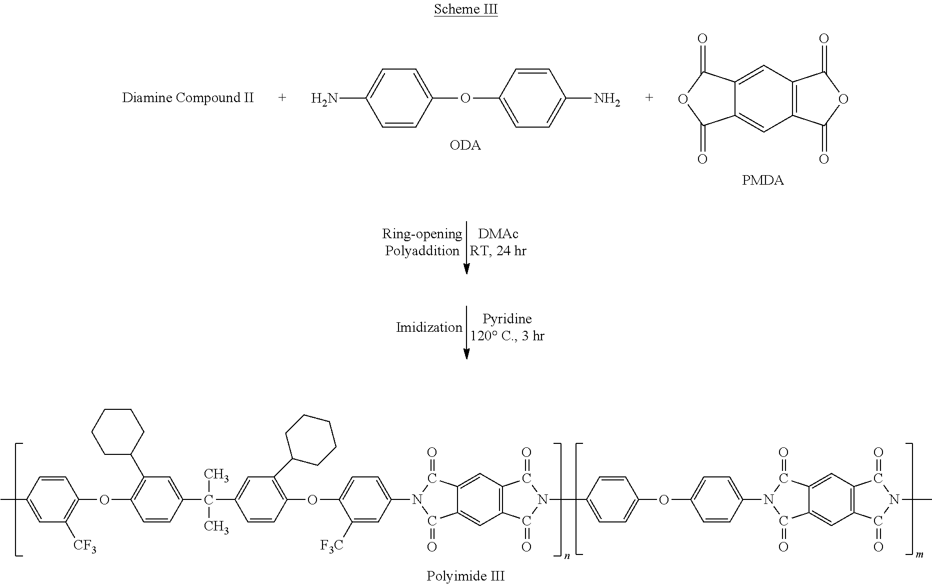

Some polyimides III were synthesized in this embodiment. The synthesis method of the polyimides III is shown in Scheme III. In this embodiment, in addition to the first aromatic diamine monomer, i.e. the diamine compound II, the second aromatic diamine monomer, 4,4′-oxydianiline (ODA), was also added. The tetracarboxylic dianhydride monomers of these polyimides III were all pyromellitic dianhydride (PMDA). The molar ratio of the two aromatic diamine monomers was varied to obtain various polyimides III containing various molar ratios of the diamine compound II and ODA.

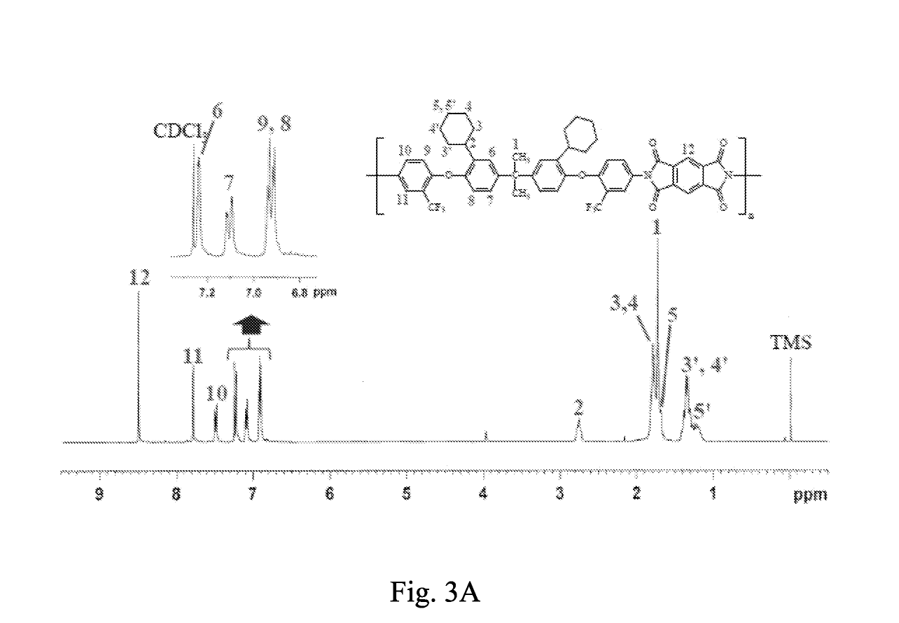

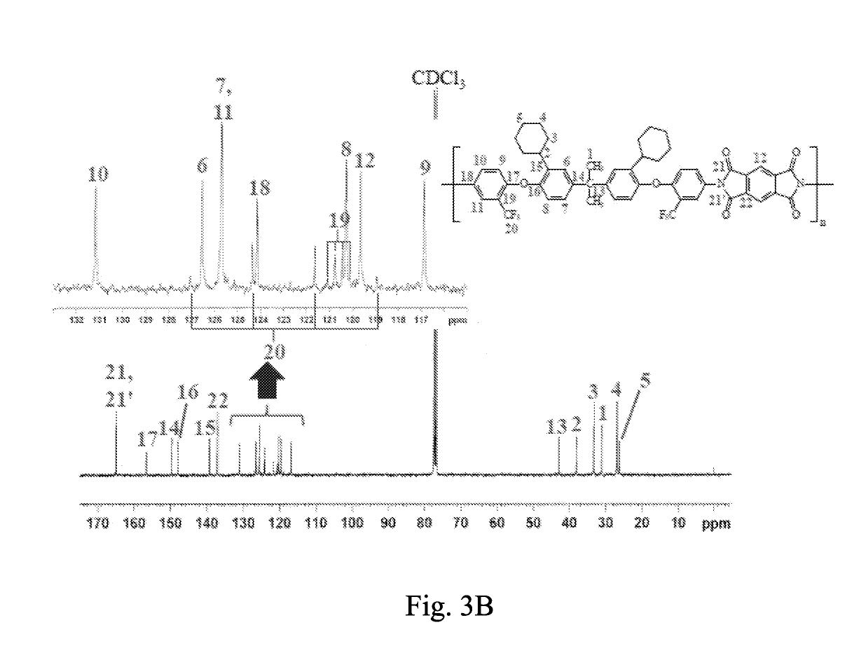

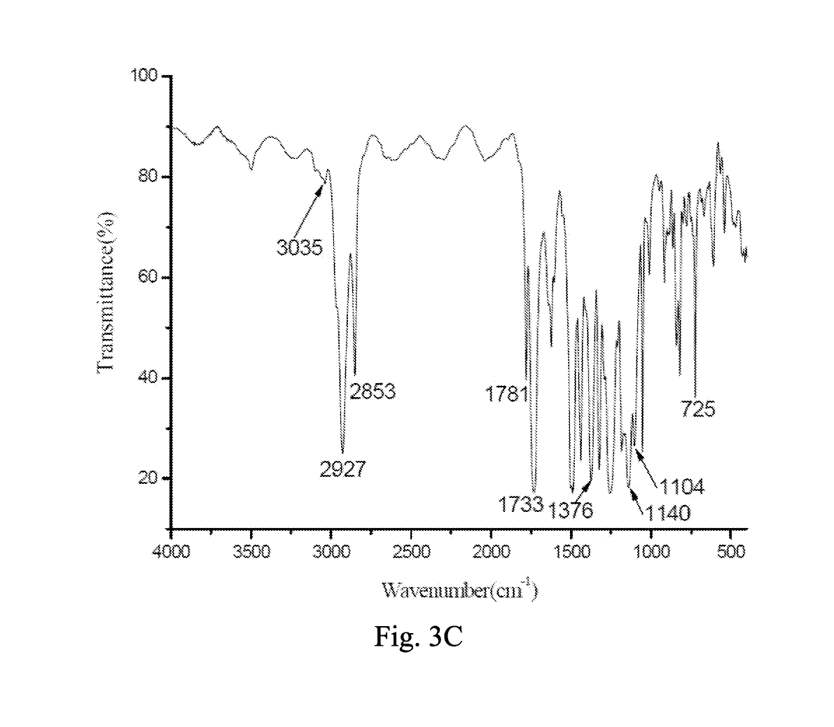

The synthesis steps of the above polyimide III using the diamine compound II and the PMDA as monomers are described below, and the obtained polyimide III was denoted as PMDA-100. 1.0 mmole of diamine compound II and 10 mL of N,N-dimethylacetamide (DMAc) were added into a two-neck flask. After completely dissolving the diamine compound II in DMAc, 1.0 mmole of PMDA was slowly added in portions. The reaction mixture was stirred at room temperature for 12 hours to perform the ring opening and polyaddition reaction to form polyamic acid (PAA) intermediate. Next, 1 mL of acetic anhydride and 0.5 mL of pyridine were added to the above solution containing the PAA intermediate, and the reaction mixture was stirred at room temperature for another 1 hour. The reaction mixture was then heated to 100° C. and stirred for another 3 hours to perform the cyclization reaction. After cooling down, the reaction solution was poured into large amount of methanol to precipitate the polyimide PMDA-100. Next, methanol and hot water was used to wash the polyimide PMDA-100. The yield of the polyimide PMDA-100 was 95%. FIGS. 3A-3C are ¹H NMR, ¹³C NMR, and IR spectra of the polyimide PMDA-100, respectively. The other spectra data are listed below.

¹H-NMR (CDCl₃): δ (ppm)=8.51 (2H, H₁₂), 7.80 (2H, H₁₁), 7.52˜7.49 (2H, H₁₀), 7.24 (2H, H₆), 7.12˜7.10 (2H, H₇), 6.94˜6.91 (4H, H₉ and H₈), 2.77 (2H, H₂), 1.80 (8H, H₃ and H₄), 1.74 (6H, H₁), 1.70 (2H, H₅), 1.43˜1.29 (8H, H_{3′} and H_{4′}), 1.26˜1.20 (2H, H_{5′}); ¹³C-NMR (CDCl₃): δ (ppm)=165.0 (C₂₁), 156.7 (C₁₇), 150.0 (C₁₆), 148.0 (C₁₄), 139.3 (C₁₅), 137.2 (C₂₂), 131.2 (C₁₀), 127.1 (C₆), 125.7 (C₁₁ and C₇), 127.1, 124.4, 121.7, 118.9 (C₂₀), 124.2 (C₁₈), 121.1, 120.8, 120.4, 120.1 (C₁₉), 120.3 (C₈), 119.7 (C₁₂), 116.9 (C₉), 43.0 (C₁₃), 38.2 (C₂), 33.4 (C₃), 31.2 (C₁), 26.9 (C₄), and 26.2 (C₅). IR spectrum (cm⁻¹): 3035 (stretching vibration of aromatic C—H), 2927 and 2853 (stretching vibrations of aliphatic C—H), 1781 and 1733 (asymmetric and symmetric stretching vibrations of imide C═O), 1376 (stretching vibration of C—N), 1104 and 725 (deformation of imide ring).

In addition, the solution of the PAA intermediate also may be coated on a substrate and then heated in a high temperature furnace (100° C. 1 hour, 150° C. 1 hour, 220° C. 1 hour, 300° C. 1 hour, and 350° C. 1 hour) to perform thermal cyclization reaction. After cooling down, a polyimide thin film may be obtained.

The synthesis steps of the polyimide III using the diamine compound II and ODA as its diamine monomer are described below, and the obtained polyimide III was denoted as PMDA-50. 1.0 mmole of the diamine compound II, 1.0 mmole of ODA, and 13 mL of DMAc were added into a two-neck flask. After completely dissolving the diamine compound II and ODA in DMAc, 2.0 mmole of PMDA was slowly added in portions. The reaction mixture was stirred at room temperature for 12 hours to perform the ring opening and polyaddition reaction to form PAA intermediate. Next, 1 mL of acetic anhydride and 0.5 mL of pyridine were added to the above solution of the PAA intermediate, and the reaction mixture was stirred at room temperature for another 1 hour. The reaction mixture was then heated to 100° C. and stirred for another 3 hours to perform the cyclization reaction. After cooling down, the reaction solution was poured into large amount of methanol to precipitate the polyimide PMDA-50. Next, methanol and hot water was used to wash the polyimide PMDA-50. The yield of the polyimide PMDA-50 was 97%. IR spectrum (cm⁻¹): 1784 and 1725 (asymmetric and symmetric stretching vibrations of imide C═O), 1377 (stretching vibration of C—N), and 1100 and 723 (deformation of imide ring).

Some basic properties of PMDA-100 and PMDA-50 are listed in the Table 1 below.

TABLE 1
Some basic properties of PMDA-100 and PMDA-50

| | Molar ratio | | | | | | |
|---|---|---|---|---|---|---|---|
| | of diamine | | | | | | |
| | monomers | | | | | | |
| | Diamine | | | | | | |
| Exam- | compound | | ^{a}ηᵢₙₕ (dL/g) | | | | |
| ples | II | ODA | PAA | PI | ^{b} M̅n̅ × 10⁻⁴ | ^{b} M̅w̅ × 10⁻⁴ | ^{c}PDI |
| PMDA- | 1 | 1 | 2.65 | Gelation | Insol- | Insol- | Insol- |
| 50 | | | | Insol- | uble | uble | uble |
| | | | | uble | | | |
| PMDA- | 1 | 0 | 0.60 | 0.55 | 4.6 | 9.0 | 1.96 |
| 100 | | | | | | | |

^{a}Inherent viscosity was measured at 30° C. for 0.5 g/dL DMAc solution of PAA or PI.
^{b}Number average molecular weight M̅n̅ and weight average molecular weight M̅w̅ of polyimides were measured by gel permeation chromatography (GPC) in DMAc.
^{c}PDI = M̅w̅/ M̅n̅

### Embodiment 3

### Synthesis of Polyimide IV

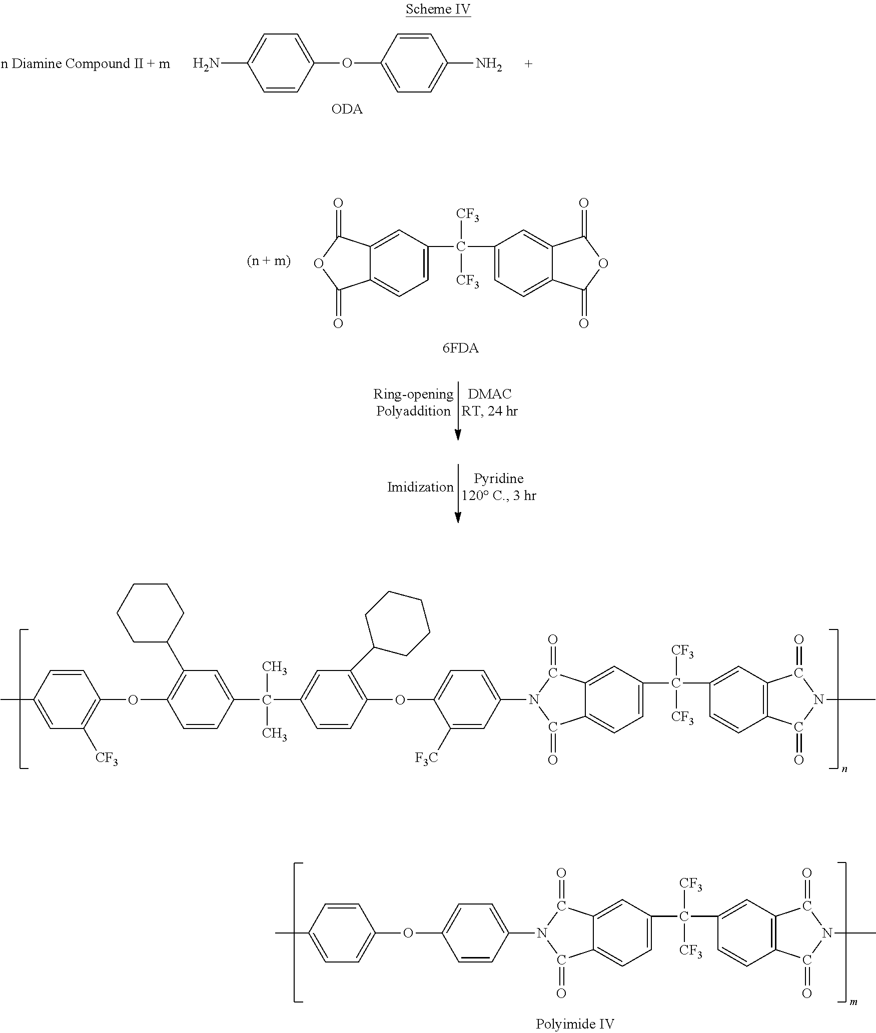

Some polyimides IV were synthesized in this embodiment. The synthesis method of the polyimide IV is shown in Scheme IV. In this embodiment, in addition to the first aromatic diamine monomer, i.e. the diamine compound II, the second aromatic diamine monomer, 4,4′-oxydianiline (ODA), was also added. The tetracarboxylic dianhydride monomers of these polyimides IV were all 4,4′-hexafluoroisopropylidene bisphthalic dianhydride (6FDA). The molar ratio of the two aromatic diamine monomers was varied to obtain various polyimides IV containing various molar ratios of the diamine compound II and ODA. Since the detailed synthesis steps of the polyimide IV are similar to the synthesis steps of the polyimide III, the only difference is the tetracarboxylic dianhydride monomer PMDA in the synthesis of the polyimide III was replaced by 6FDA in the synthesis of the polyimide IV.

Some basic properties of the synthesized polyimides IV with various molar ratios of aromatic diamine monomers are listed in Table 2 below. The number in the names of the polyimides IV denoted the added molar percentage of the diamine compound II of the total added aromatic diamine monomers.

TABLE 2
Some basic properties of synthesized polyimide IV

| | Molar ratio of | | | | | | |
|---|---|---|---|---|---|---|---|
| | diamine | | | | | | |
| | monomers | | | | | | |
| | Diamine | | | | | | |
| | com- | | | | | | |
| | pound | | ^{a}ηᵢₙₕ (dL/g) | | | | |
| Examples | II | ODA | PAA | PI | ^{b} M̅n̅ × 10⁻⁴ | ^{b} M̅w̅ × 10⁻⁴ | ^{c}PDI |
| 6FDA- | 0 | 1 | 0.82 | 0.65^{d} | 4.4 | 8.9 | 2.01 |
| 0 | | | | | | | |
| 6FDA- | 1 | 9 | 0.72 | 0.62 | 5.2 | 9.9 | 1.88 |
| 10 | | | | | | | |
| 6FDA- | 1 | 3 | 0.70 | 0.61 | 5.7 | 11.3 | 1.99 |
| 25 | | | | | | | |
| 6FDA- | 1 | 1 | 0.74 | 0.64 | 7.2 | 14.0 | 1.96 |
| 50 | | | | | | | |
| 6FDA- | 1 | 0 | 0.64 | 0.55 | 6.4 | 10.8 | 1.69 |
| 100 | | | | | | | |

^{a}Inherent viscosity was measured at 30° C. for 0.5 g/dL DMAc solution of PAA or PI.
^{b}Number average molecular weight M̅n̅ and weight average molecular weight M̅w̅ of polyimides were measured by gel permeation chromatography (GPC) in DMAc.
^{c}PDI = M̅w̅/ M̅n̅
^{d}dissolved in NMP

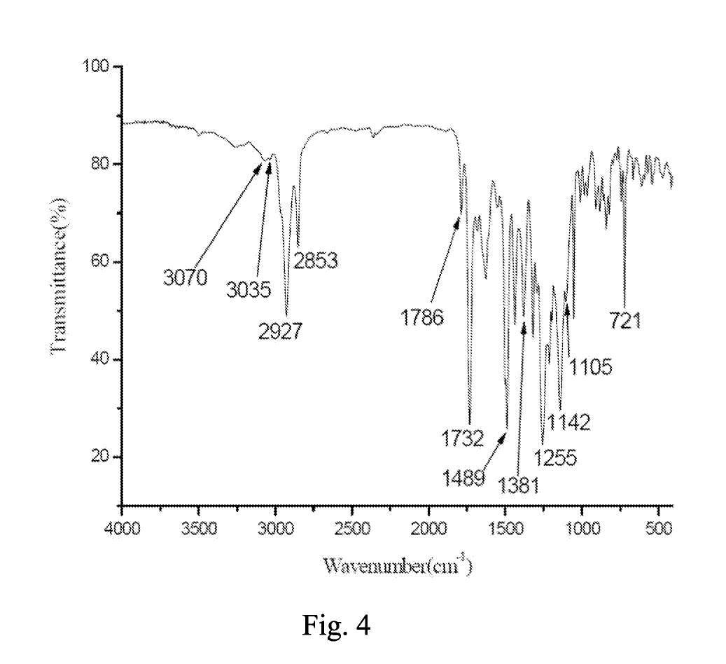

FIG. 4 is IR spectrum of 6FDA-100. The vibration peaks (cm⁻¹) of IR spectrum includes 3035 (stretching vibration of aromatic C—H), 2927 and 2853 (stretching vibrations of aliphatic C—H), 1786 and 1732 (asymmetric and symmetric stretching vibrations of imide C═O), 1381 (stretching vibration of C—N), and 1105 and 721 (deformation of imide ring).

### Embodiment 4

### Solubility Test of Polyimides

In this embodiment, the solubility of various polyimides in various organic solvents was tested. The solubility measurements were performed by dissolving 10 mg polyimide in 1 mL organic solvent including N-methylpyrrolidone (NMP), dimethyl acetamide (DMAc), dimethylformamide (DMF), dimethyl sulfoxide (DMSO), tetrahydrofuran (THF), pyridine (py), m-cresol, and dichloromethane (DCM).

First, the effect of trifluoromethyl group (—CF₃) and cyclohexyl group on the solubility of the tested polyimides was studied. Therefore, the solubility of polyimide 6FDA-100 and PMDA-100 were compared and the obtained test results are listed in Table 3 below. The comparison examples 2 and 3 are cited from J. Appl. Polym. Sci. 2005, 95, 922-935.

The symbol “++” in Table 3 means that the polyimide may be completely dissolved at room temperature. The symbol “+” in Table 3 means that the polyimide may be completely dissolved at 70° C. The symbol “+−” in Table 3 means that the polyimide may be partially dissolved at 70° C. The symbol “−” in Table 3 means that the polyimide cannot be dissolved at 70° C.

TABLE 3
Effect of trifluoromethyl group (—CF₃) and cyclohexyl group on the solubility of polyimide

| | | | Comparison example | | |
|---|---|---|---|---|---|
| Sample | ^{a}6FDA-100 | ^{b}PMDA-100 | ^{c}1 | ^{d}2 | ^{e}3 |
| NMP | ++ | ++ | ++ | ++ | ++ |
| DMAc | ++ | ++ | ++ | ++ | ++ |
| DMF | ++ | ++ | ++ | ++ | + |
| DMSO | ++ | + | +− | + | + |
| THF | ++ | ++ | ++ | ++ | ++ |
| py | ++ | ++ | ++ | ++ | ++ |
| m-cresol | ++ | ++ | ++ | + | + |
| DCM | ++ | +− | ++ | ++ | ++ |

a 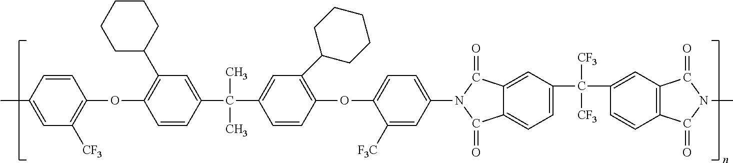
b 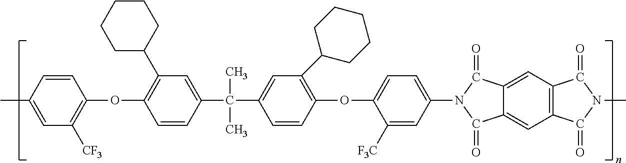
c 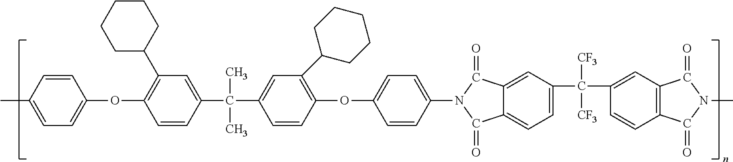
d 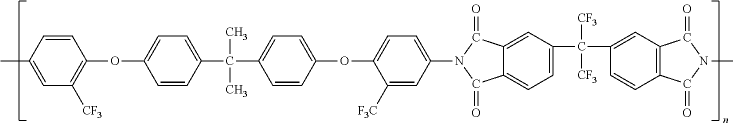
e

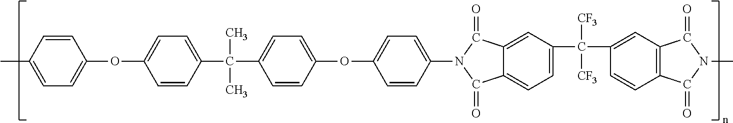

From Table 3, it may be known that the structure of the polyimide 6FDA-100 have cyclohexyl, trifluoromethyl, and 6FDA groups. Therefore, the polyimide 6FDA-100 can easily destroy the steric regularity in the polymer chain. Hence, the solubility of the polyimide 6FDA-100 was excellent, it could be soluble in all of the tested solvents at room temperature.

The solubility of the polyimide PMDA-100 in DMSO and DCM was poorer than 6FDA-100 because the polyimide 6FDA-100 has more fluorine content than PMDA-100 to decrease the steric regularity of polymer chain.

Comparing the polyimide 6FDA-100 and the comparison example 1, the daimine monomer of the comparison example 1 did not have trifluoromethyl group, and the steric regularity thereof was thus increased. Therefore, the solubility of the comparison example 1 in DMSO is poorer than the polyimide 6FDA-100. The comparison example 1 only could be partially dissolved in DMSO at 70° C.

Comparing the comparison examples 2 and 3, the diamine monomer of the comparing example 3 did not have trifluoromethyl group, and the steric regularity thereof was thus increased. Therefore, the solubility of the comparison example 3 was poor, and it could dissolve in DMF at 70° C.

Comparing the polyimide 6FDA-100 and the comparison example 2, the comparison example 2 did not have cyclohexyl group to decrease the steric regularity of the comparison example 2. Therefore, the solubility of the comparison example 2 in DMSO and m-cresol is poorer than the polyimide 6FDA-100. The comparison example 2 could dissolve in DMSO and m-cresol at 70° C.

Comparing the comparison examples 3 and 1, the comparison example 3 does not have cyclohexyl groups to decrease the steric regularity of the comparison example 3. Therefore, the solubility of the comparison example 3 in DMF and m-cresol is poorer than the comparison example 1. The comparison example 3 could dissolve in DMF and m-cresol at 70° C. However, the solubility of the comparison example 3 in DMSO was better than the comparison example 1.

Accordingly, increasing the content of the cycohexyl and trifluoromethyl groups of the polyimides can indeed increase the solubility of the polyimides in organic solvents.

Next, the solubility of various polyimides IV containing various amount of the diamine compound II in various organic solvents was tested. The testing method and the denoted symbols are the same as the above experiments. In addition, 10 mg/1 mL sulfuric acid solution was also used as a tested solvent. The obtained results were listed in Table 4. The denoted symbols in Table 4 have the same meanings as the symbols in Table 3, and the explanations are hence omitted here.

TABLE 4
Solubility of various polyimides IV in various solvents

| Sample | 6FDA-0 | 6FDA-10 | 6FDA-25 | 6FDA-50 | 6FDA-100 |
|---|---|---|---|---|---|
| NMP | ++ | ++ | ++ | ++ | ++ |
| DMAc | + | ++ | ++ | ++ | ++ |
| ^{a}DMAc | + | + | ++ | ++ | ++ |
| DMF | +− | ++ | ++ | ++ | ++ |
| DMSO | + | ++ | ++ | ++ | ++ |
| THF | +− | ++ | ++ | ++ | ++ |
| py | ++ | ++ | ++ | ++ | ++ |
| m-cresol | ++ | ++ | ++ | ++ | ++ |
| DCM | +− | ++ | ++ | ++ | ++ |
| Sulfuric | ++ | ++ | ++ | ++ | ++ |
| acid | | | | | |

^{a}The test method was dissolving 100 mg of polyimide in 1 mL solvent.

Accordingly, when the test method was performed by dissolving 10 mg of polyimide in 1 mL of solvent and the addition amount of the diamine compound II was more than 10 mole %, the obtained polyimide IV may be dissolved in all tested solvents at room temperature. When the test method was performed by dissolving 100 mg of polyimide in 1 mL of solvent, the polyimide 6FDA-10 also may be completely dissolved at 70° C., and polyimide 6FDA-25, 6FDA-50, and 6FDA-100 may be completely dissolved in the tested organic solvents at room temperature. This result shows that polyimides using the diamine compound II as one diamine monomer have excellent solubility, and hence are suitable to be used for coating process.

### Embodiment 5

### Thermal Properties of Polyimide

In this embodiment, the various thermal properties of the polyimides III and IV were evaluated by DSC and TGA. The obtained results are shown in Table 5.

TABLE 5
Thermal properties of various polyimides III and IV

| | ^{a}Tg (° C.) | | | |
|---|---|---|---|---|
| | PAA | PAA | | |
| | Thermal | Chemical | | |
| Sample | cyclization | cyclization | ^{b}Td_{10%} (° C.) | ^{c}R_{W800} (%) |
| ^{d}PMDA-0 | 362 | No data | 601 | 54 |
| PMDA-50 | 285 | gelation | 479 | 53 |
| PMDA-100 | 245 | 234 | 471 | 30 |
| ^{d}6FDA-0 | 296 | No data | 538 | 56 |
| 6FDA-0 | 306 | 295 | 554 | 52 |
| 6FDA-10 | 311 | 283 | 541 | 55 |
| 6FDA-25 | 279 | 268 | 497 | 47 |
| 6FDA-50 | 250 | 242 | 490 | 39 |
| 6FDA-100 | 229 | 220 | 478 | 26 |

^{a}Glass transition temperature (Tg) was measured by differential scanning calorimetry (DSC), and the heating rate was 10° C./min.
^{b}The thermal decomposition temperature at 10 wt % loss (Td_{10%}) was measured by thermogravimetric analysis (TGA), and the heating rate was 20° C./min.
^{c}The sample's residue weight percentage at 800° C. (R_{W800}) was measured by TGA.
^{d}Data was cited from J. Appl. Polym. Sci. 2010, 117, 1144-1155.

From the data listed in Table 5, it may be known that the thermal decomposition temperature at 10 wt % loss (Td_{10%}) and the glass transition temperature (Tg) was decreased as the content of the diamine compound II in the polyimide was increased. This result shows that the thermal stability was decreased as the content of the diamine compound II in the polyimide was increased. This phenomenon may be caused by the cyclohexyl groups of the diamine compound II. Therefore, the sample's residue weight percentage at 800° C. (R_{W800}) was also decreased as the content of the diamine compound II in the polyimide was increased.

### Embodiment 6

### Optical Properties of Polyimides

In this embodiment, transmittance of UV to visible light (200-800 nm) of various polyimides was measured. First, the effect of trifluoromethyl group and cyclohexyl group on the transmittance of polyimide was studied. The obtained test results are listed in Table 6. The comparison examples 2 and 3 are cited from J. Appl. Polym. Sci. 2005, 95, 922-935. In Table 6, the cut-off wavelength was defined as the wavelength that has a transmittance smaller than 1%.

The transmittance of each sample was close to zero in the UV region of short wavelength and increased in the long wavelength region having a wavelength longer than the cut-off wavelength. Since the broadest wavelength region of the visible light is 380-780 nm, the polyimide film is more light-colored as the cut-off wavelength is shorter.

TABLE 6
Transmittance of various polyimides

| | | | wavelength of | |
|---|---|---|---|---|
| Sample | thickness (μm) | cut-off wavelength (nm) | 80% transmittance (nm) | transmittance at 550 nm (%) |
| ^{a}6FDA-100 | 45 | 339 | 425 | 90 |
| ^{b}Comparison example 2 | 36 | 365 | No Data | No Data |
| ^{c}Comparison example 3 | 42 | 375 | No Data | No Data |
| ^{d}PMDA-100 | 50 | 410 | 496 | 86 |

a 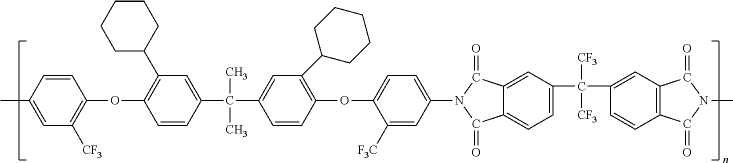
b 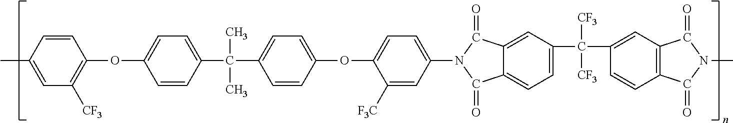
c 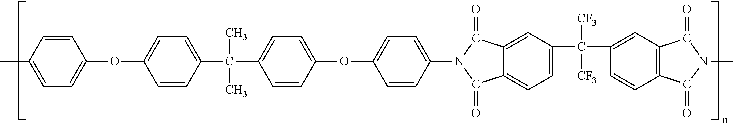
d

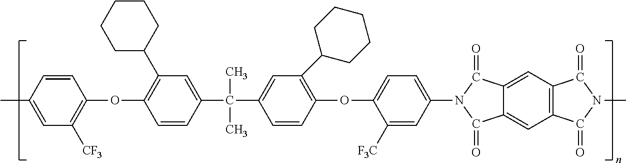

From the result shown in Table 6, it may be known that the polyimide 6FDA-100 has the shortest cut-off wavelength (339 nm) and the wavelength at 80% transmittance was also shorter (425 nm). Therefore, the visual color of the polyimide 6FDA-100 is more transparent and colorless than the polyimide PMDA-100, the comparison examples 2 and the comparison examples 3. The polyimide PMDA-100 has the longest cut-off wavelength (410 nm). Therefore, the visual color of the polyimide PMDA-100 is pale yellow.

Introducing the cyclohexyl and trifluoromethyl groups into the polyimide chain (the polyimide 6FDA-100) has shorter cut-off wavelength and shorter wavelength at 80% transmittance than the polyimide PMDA-100, the comparison examples 2 and the comparison examples 3.

Next, the transmittance of various polyimides IV containing various amounts of the diamine compound II was measured. The results are shown in Table 7. The measuring method and the meaning of the physical properties in Table 7 are the same as the Table 6.

TABLE 7
Transmittance of various polyimides IV

| | thick- | cut-off | | |
|---|---|---|---|---|
| | ness | wavelength | wavelength at 80% | transmittance |
| Sample | (μm) | (nm) | transmittance (nm) | at 550 nm (%) |
| 6FDA-0 | 25 | 379 | 484 | 83 |
| 6FDA-10 | 23 | 377 | 444 | 87 |
| 6FDA-25 | 23 | 374 | 440 | 88 |
| 6FDA-50 | 24 | 363 | 427 | 88 |
| 6FDA-100 | 28 | 320 | 418 | 90 |

From the results shown in Table 7, it may be known that the cut-off wavelength and the wavelength at 80% transmittance were shorter, and the transmittance at 550 nm was higher when the molar fraction of the diamine compound II used for synthesizing the polyimide IV was greater. This result indicated that the polyimide IV may be more close to colorless as the molar fraction of the diamine compound II used for synthesizing the polyimide IV was greater, and the polyimide IV was thus more suitable to be applied on optoelectronic products.

In light of foregoing, after adding the diamine compound II into the conventional polyimides, the solubility of the polyimides containing the diamine compound II in organic solvents may be increased, as well as the color of these polyimides may be more close to transparent and colorless. Therefore, the polyimides containing the diamine compound II may be more easily dissolved in organic solvents to form polyimide solution, which can facilitate coating process. The obtained more transparent and colorless polyimides containing the diamine compound II are more suitable to be used in optoelectronic products.

All the features disclosed in this specification (including any accompanying claims, abstract, and drawings) may be replaced by alternative features serving the same, equivalent or similar purpose, unless expressly stated otherwise. Thus, each feature disclosed is one example only of a generic series of equivalent or similar features.

What is claimed is:

1. A dinitro compound I having a chemical structure shown below:

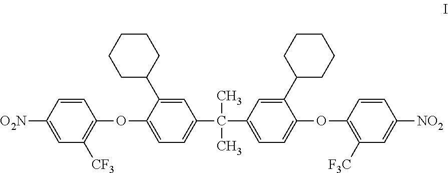 [SMILES: CC(C)(c1ccc(Oc2ccc([N+](=O)[O-])cc2C(F)(F)F)c(C2CCCCC2)c1)c1ccc(Oc2ccc([N+](=O)[O-])cc2C(F)(F)F)c(C2CCCCC2)c1]

2. A diamine compound II having a chemical structure shown below:

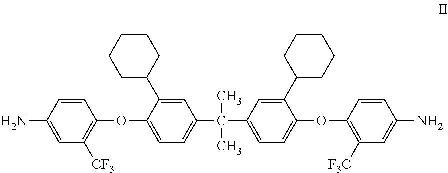 [SMILES: CC(C)(c1ccc(Oc2ccc(N)cc2C(F)(F)F)c(C2CCCCC2)c1)c1ccc(Oc2ccc(N)cc2C(F)(F)F)c(C2CCCCC2)c1]

3. A polyimide, wherein monomers of the polyimide comprises a first aromatic diamine monomer having a chemical structure shown below, 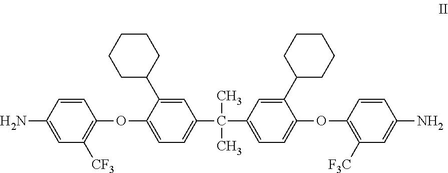 [SMILES: CC(C)(c1ccc(Oc2ccc(N)cc2C(F)(F)F)c(C2CCCCC2)c1)c1ccc(Oc2ccc(N)cc2C(F)(F)F)c(C2CCCCC2)c1]
and a tetracarboxylic dianhydride monomer having a chemical structure of

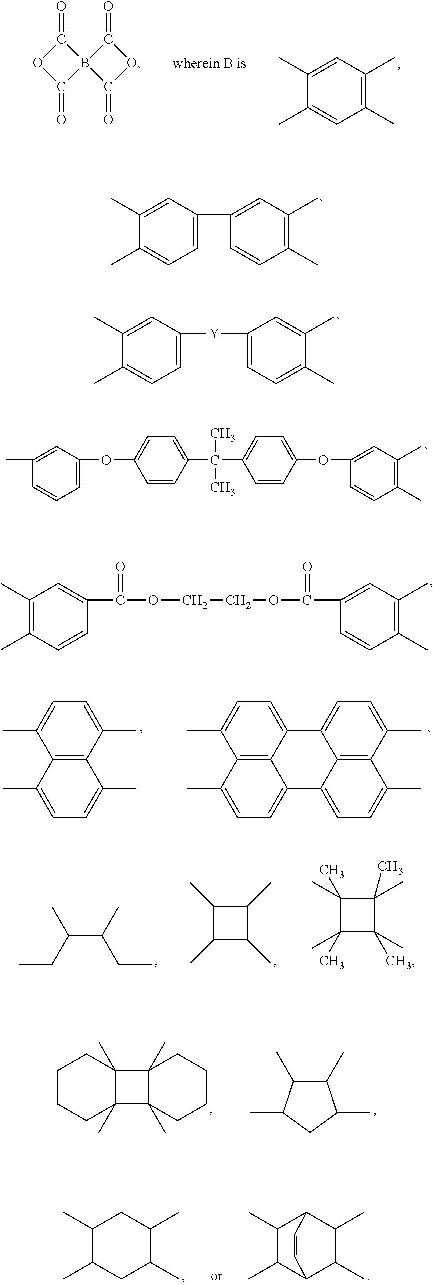

4. The polyimide of claim 3, wherein the

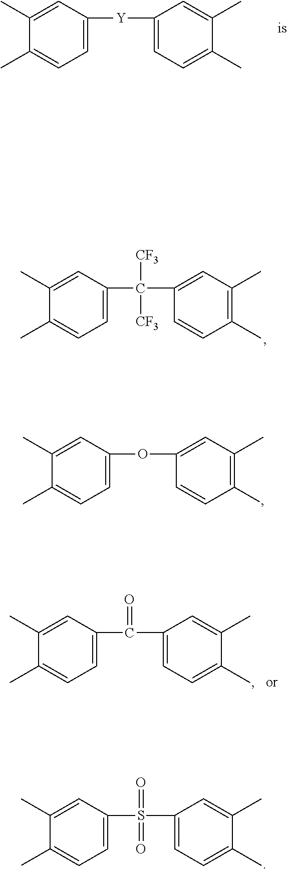

5. The polyimide of claim 3, wherein the monomers of the polyimide further comprises a second aromatic diamine monomer H₂N-A-NH₂, wherein A is

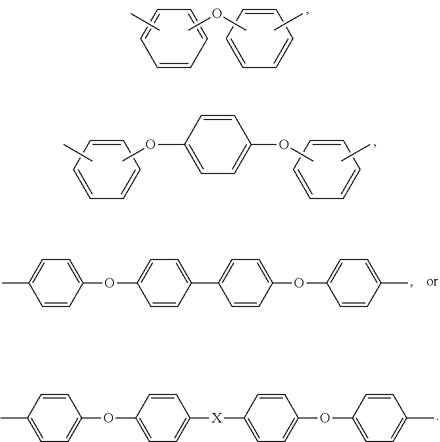

6. The polyimide of claim 5, wherein the

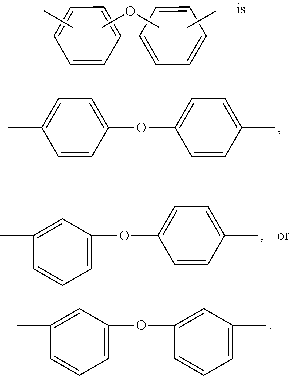

7. The polyimide of claim 5, wherein the

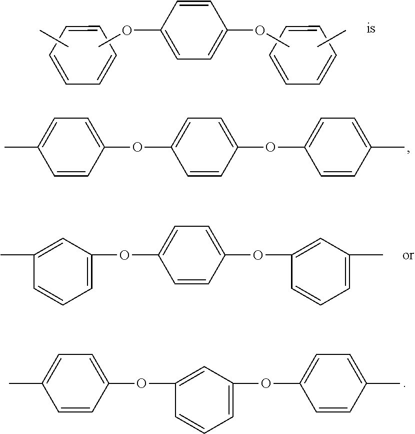

8. The polyimide of claim 5, wherein the

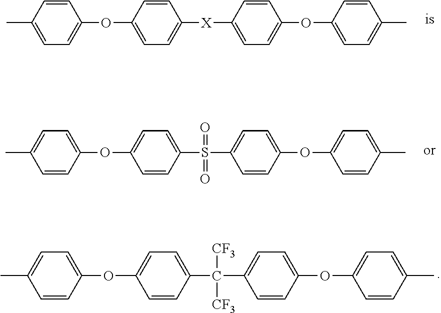

9. The polyimide of claim 5, wherein a molar ratio of the second aromatic diamine monomer to the first aromatic diamine monomer is 0-99.

10. The polyimide of claim 5, wherein the B of the tetracarboxylic dianhydride monomer is

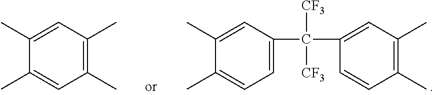

11. The polyimide of claim 5, wherein the second aromatic diamine monomer is 4,4′-oxydianiline.

12. The polyimide of claim 3, wherein the B of the tetracarboxylic dianhydride monomer is [chemical structure]
